(19) United States
(12) Plant Patent Application Publication
(10) Pub. No.: US 2004/0055065 P1
(43) Pub. Date: Mar. 18, 2004

(54) Petunia plant named 'Kakegawa S66'

(76) Inventor: Masao Bessho, Kakegawa City (JP)

Correspondence Address: JONDLE & ASSOCIATES P.C., 9085 EAST MINERAL CIRCLE, SUITE 200, CENTENNIAL, CO, 80112, US

(21) Appl. No.: 10/243,993

(22) Filed: Sep. 13, 2002

(51) Int. Cl. A01H 5/00

(52) U.S. Cl. PLT/356

(57) ABSTRACT

A Petunia cultivar particularly distinguished by its pale lavender flower color with purple streaking at the petal base.

### GENUS AND SPECIES

Petunia hybrida

### VARIETY DENOMINATION

‘Kakegawa S66’

### BACKGROUND OF THE NEW PLANT

The present invention comprises a new and distinct cultivar of Petunia, botanically known as Petunia hybrida, and hereinafter referred to by the cultivar name ‘Kakegawa S66’. ‘Kakegawa S66’ originated from a hybridization made in 1997 in Kakegawa, Japan. The male parent was a phenotypically fixed F₃ selection from a cross made in 1994 between two breeding lines named 4UK-1 and P-1a. The female parent of ‘Kakegawa S66’ was an F₅ selection known as 89S-829-1a-1a-1a-1, originating from a dwarf multiflora breeding gene pool. F₁ seed was obtained from this cross in 1997.

Two hundred F₁ plants from the 1997 cross were transplanted to the field in Salinas, Calif. during the summer of 1998. Seven lines were selected for further evaluation and vegetatively propagated. The seven lines were propagated again in 1999 and evaluated for trueness to type and ease of propagation. Final selection of one line was made in Salinas, Calif. during the summer of 1999. The line was established as ‘Kakegawa S66’, and determined to have its characteristics firmly fixed.

‘Kakegawa S66’ has been found to retain its distinctive characteristics after three years and five cycles of vegetative propagation and this novelty is firmly fixed. The variety has demonstrated stability during this time and has no inherent variation or off-types.

### DESCRIPTION OF PHOTOGRAPH

This new Petunia plant is illustrated by the accompanying photograph which shows blooms, and foliage of the plant in full color, the colors shown being as true as can be reasonably obtained by conventional photographic procedures.

FIG. 1 shows the mature inflorescence;

FIG. 2 shows the entire plant approximately twelve weeks after transplanting a rooted cutting in a six-inch pot.

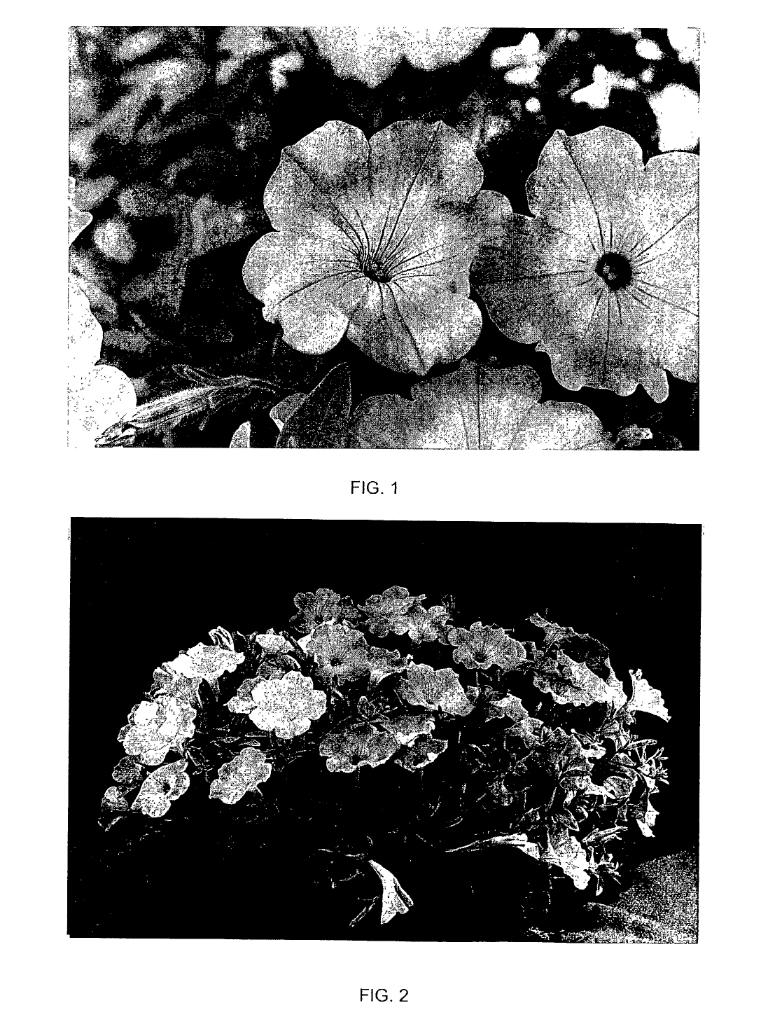

### DESCRIPTION OF THE NEW CULTIVAR

The following detailed descriptions set forth the distinctive characteristics of ‘Kakegawa S66’. The data which defines these characteristics were collected from asexual reproductions carried out in Salinas, Calif. Data was collected on plants in 15 cm diameter pots about twelve weeks after rooted cuttings were transplanted. Color references are primarily to the RHS Colour Chart of The Royal Horticultural Society of London (RHS).

### DESCRIPTION OF THE NEW PLANT

Classification:

Botanical.—Petunia hybrida.

Commercial.—Petunia.

Parentage:

Female parent.—breeding line 89S-829-1a-1a-1a-1 (not patented).

Male parent.—F₃ selection from cross of breeding lines 4UK-1 and P-1a (not patented).

Environmental conditions for plant growth: Plants were propagated from vegetative cuttings, and grown individually in 15 cm diameter plastic pots in a glass greenhouse located in Salinas, Calif. Pots contained a peat moss-based growing medium. Soluble fertilizer containing 18% nitrogen, 8% phosphorus and 18% potassium was applied in four, daily irrigations. The fifth irrigation was made with non-fertilized water. Pots were top-dressed with a slow release fertilizer containing 18% nitrogen, 8% phosphorus and 18% potassium. The typical average air temperature was 24 C.

Growth:

Habit.—branching, mounding.

Form.—descending.

Plant Size.—91 cm total diameter and 76 cm total height when three plants are grown in a 50 cm diameter hanging basket for 7-8 weeks.

Flowering habit.—indeterminate.

Time to bloom from propagation.—4-6 weeks after rooting when grown in 10-15 cm diameter plastic pots.

Life Cycle.—annual.

Stems:

Color.—yellow-green (RHS 145A).

Description.—round, pubescent.

Diameter.—2 to 3 mm.

Internode length.—4.0 cm.

Leaves:

Arrangement.—opposite.

Apex.—mucronate.

Base.—oblique.

Color.—upper surface is green (RHS 137B) and lower surface is green (RHS 138B).

Margin.—entire.

Size.—length is 4.8 cm at full expansion and width is 2.5 cm at full extension.

Shape.—ovate.

Texture.—coarse.

Venation.—pinnate.

Pubescence.—present, clear.

Flowers:

Bud color.—Yellow-green (RHS 149B).

Bud shape.—Ovate.

Bud surface.—Extensively pubescent.

Calyx.—5 sepals; 2 cm×8 mm (length×width); free.

Corolla.—5 petals, fused.

Flower diameter.—6.0-6.5 cm.

Fragrant.—yes.

Inflorescence type.—solitary.

Pistil.—compound.

Ovary.—superior, parietal placentation.

Stamens.—5 total with two long and three short; white (RHS 155C).

Peduncle.—1.5 cm×1 mm (length×width); pubescent.

Petal color.—Limbs: upper — purple (RHS 77B); lower — purple (RHS 76B) with yellow-green (RHS 145B) veins. Tube — Inner — green (RHS 144D) with purple (RHS 77A) veins; Outside is yellow-green (RHS 144B) with green (RHS 140A) veins.

Petal pubescence.—absent.

Tube throat diameter.—7 mm.

Pollen color.—White (RHS 155C).

Produces seed.—Yes.

Disease and insect resistance: No susceptibility to diseases or insects noted to date.

Comparison with known cultivars: ‘Kakegawa S66’ is a distinct variety of petunia owing to its pale lavender flower color and purple streaking at the petal base. ‘Kakegawa S66’ is most similar to the variety ‘Cascadia Charme’ however there are numerous differences as shown in the table below.

| Characteristics | ‘Kakegawa S66’ | ‘Cascadia Charme’ |
|---|---|---|
| Petal Shape | Rounded | Pointed |
| Flow Vein Color | Yellow-green (RHS 145B) | Yellow-green (RHS |
| | with purple (RHS 76A) | 145B) with no streaks |
| | streaks | |
| Flower Diameter | 6.0-6.5 cm | 7.0-8.5 cm |
| Leaf Width | 2.5 cm | 5.5 cm |
| Leaf Length | 4.8 cm | 7.0-8.5 cm |

I claim:

1. A new and distinct cultivar of Petunia as shown and described herein.
